(19) United States
(12) Patent Application Publication
(10) Pub. No.: US 2026/0139947 A1
(43) Pub. Date: May 21, 2026

(54) SYSTEMS AND METHODS FOR WEARABLE MOTION TRACKING

(71) Applicant: MASSACHUSETTS INSTITUTE OF TECHNOLOGY, Cambridge, MA (US)

(72) Inventors: Gengxi Lu, Allston, MA (US); Xuanhe Zhao, Cambridge, MA (US)

(21) Appl. No.: 19/387,138

(22) Filed: Nov. 12, 2025

Related U.S. Application Data
(60) Provisional application No. 63/721,387, filed on Nov. 15, 2024.

(51) Int. Cl. G01C 21/16; A61B 8/00

(52) U.S. Cl. CPC G01C 21/165; A61B 8/4227; A61B 8/4472

(57) ABSTRACT

System and methods are provided for body motion tracking. The system includes one or more ultrasound systems configured to be worn by a subject and to acquire ultrasound data during motion of a first location on the subject. The system also includes one or more inertia measurement unit (IMU) systems configured to be worn on a second location on the subject and to measure inertia data associated with motion of the second location and a control system configured to integrate the ultrasound data and the inertia data to generate position data, the position data characterizing a motion of one of the first or second location on the subject.

### CROSS-REFERENCE TO RELATED APPLICATIONS

This application is based on, claims priority to, and incorporates herein by reference for all purposes, U.S. Provisional Patent Application No. 63/721,387 filed on Nov. 15, 2024.

### STATEMENT REGARDING FEDERALLY SPONSORED RESEARCH

This invention was made with government support under 1R01HL153857-01 and 1R01HL167947-01 awarded by the National Institutes of Health, under EFMA-1935291 awarded by the National Science Foundation, and under PR200524P1 awarded by the Department of Defense. The government has certain rights in the invention.

### BACKGROUND

Performing full-body tracking using wearable sensors is increasingly important, particularly in areas like healthcare, sports, rehabilitation, virtual and augmented reality, and human-computer interaction. However, fully-body motion tracking is particularly challenging due to the wide range of motion scales across the whole body. Body tracking is typically achieved using cameras. However, this requires large and complex setups that are limited by the camera frame or multiple camera frames. These systems typically do not provide portability, limiting the physical space in which motion can be tracked. Moreover, the camera view can become obstructed. Thus, many motions cannot be accurately tracked with a reasonable number of cameras as some anatomical regions can become hidden from view of the cameras over the course of the movement. For example, in tracking a pinching gesture, the thumb and index finger may be blocked by the palm in some viewing angles. As another example, tracking motion of a hand while using a tool (e.g., scissors) can be challenging when the tool obstructs the camera view. Thus, new methods are desired that provide reliable body tracking over different scales of motion (e.g., combined hand and arm).

### SUMMARY

The present disclosure overcomes the aforementioned drawbacks by providing systems and methods for wearable full-body motion tracking. The systems and methods may allow for hand tracking. Such body tracking can be described as position data, which may be referred to as motion data, pose data, or configuration data. Position data can be described by degrees of freedom associated with the anatomically relevant degrees of motion.

In accordance with one aspect of the disclosure, a body motion tracking system is provided that includes one or more ultrasound systems configured to be worn by a subject and to acquire ultrasound data during motion of a first location on the subject and one or more inertia measurement unit (IMU) systems configured to be worn on a second location on the subject and to measure inertia data associated with motion of the second location. The system also includes a control system configured to integrate the ultrasound data and the inertia data to generate position data, the position data characterizing a motion of one of the first or second location on the subject.

In accordance with another aspect of the disclosure, a human motion tracking system is provided that includes a wearable ultrasound system configured to acquire ultrasound data from a first structure of a body of a wearer, a wearable inertial measurement unit (IMU) system configured to measure inertia data associated with a second structure of the body of the wearer, and one or more control systems storing a first algorithm and a second algorithm. The first algorithm is configured to receive the ultrasound data from the wearable ultrasound system and determine a pose of the wearer's first structure based on the ultrasound data. The second algorithm is configured to receive the inertia data from the IMU system and determine a position of the wearer's second structure based on the inertia data. Further, the one or more control systems are further configured to integrate the pose of the wearer's first structure with the position of the wearer's second structure to generate position data of an extended region of the body of the wearer comprising the first structure and the second structure.

In accordance with yet another aspect of the disclosure, a method of is provided for determining a pose of a portion of a user. The method includes imaging an internal structure of a body of the user using a wearable ultrasound system, inputting ultrasound data from the wearable ultrasound system to a machine learning algorithm, and determining a pose of a first structure of the user by the machine learning algorithm, based on the ultrasound data. The method also includes measuring inertia data using a wearable IMU system coupled to the second structure of the user, inputting the inertia data from the IMU system to an algorithm, and determining a pose of the second structure of the user by the algorithm, based on the inertia data. The method also includes integrating the pose of the first structure of the user and the pose of the second structure of the user to determine the pose of the portion of the user.

These aspects are nonlimiting. Other aspects and features of the systems and methods described herein will be provided below.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 is a block diagram of an example body-tracking system.

FIG. 2 is a block diagram of an example ultrasound system.

FIG. 3 is a block diagram of an example inertia measurement unit system.

FIG. 4A illustrates an example process that can be used to integrate configuration data on different motion scales.

FIG. 4B shows results from an example body-tracking system including an ultrasound wristband system on a wrist and an inertia measurement unit system on a forearm of a subject.

FIG. 5 illustrates components of an example body-tracking system that can be used for full-body tracking in accordance with the present disclosure.

FIG. 6 is a block diagram of an example tracking system that can implement the methods of the present disclosure.

FIG. 7 is a block diagram of example components that can implement the system of FIG. 6.

FIG. 8 illustrates steps of an example process for applying a machine learning algorithm to determine position data from sensor data.

FIG. 9 illustrates steps of an example process for training a machine learning algorithm to determine position data from sensor data.

FIG. 10A illustrates a comparison between a comparative example and an example of the present disclosure.

FIG. 10B illustrates a comparison between a comparative example and an example of the present disclosure.

FIG. 10C illustrates a comparison between a comparative example and an example of the present disclosure.

FIG. 10D illustrates a comparison between a comparative example and an example of the present disclosure.

FIG. 11A illustrates an example design of a wearable high-resolution ultrasound probe in accordance with the present disclosure.

FIG. 11B illustrates an example design of a wearable high-resolution ultrasound probe in accordance with the present disclosure.

FIG. 11C illustrates an example design of a wearable high-resolution ultrasound probe in accordance with the present disclosure.

FIG. 12A illustrates an example of a wearable high-resolution ultrasound probe in accordance with the present disclosure, coupled to the wrist via an ultrasound couplant.

FIG. 12B illustrates an example of a wearable high-resolution ultrasound probe in accordance with the present disclosure, coupled to the wrist via an ultrasound couplant.

FIG. 12C illustrates an example of a wearable high-resolution ultrasound probe in accordance with the present disclosure, coupled to the wrist via an ultrasound couplant.

FIG. 13 illustrates an example of the manner in which ultrasound regions characterize the configurations of the five fingers and the palm, in accordance with the present disclosure.

FIG. 14A illustrates the principle and performance of an example of an ultra-dexterous virtual hand in accordance with the present disclosure.

FIG. 14B illustrates the principle and performance of an example of an ultra-dexterous virtual hand in accordance with the present disclosure.

FIG. 14C illustrates the principle and performance of an example of an ultra-dexterous virtual hand in accordance with the present disclosure.

FIG. 14D illustrates the principle and performance of an example of an ultra-dexterous virtual hand in accordance with the present disclosure.

FIG. 14E illustrates the principle and performance of an example of an ultra-dexterous virtual hand in accordance with the present disclosure.

FIG. 14F illustrates the principle and performance of an example of an ultra-dexterous virtual hand in accordance with the present disclosure.

FIG. 15 illustrates the architecture of an artificial intelligence model in accordance with the present disclosure.

### DETAILED DESCRIPTION

The present disclosure may be implemented on or with the use of computing devices including control units or control systems, processors, and/or memory elements in some examples. As used herein, a “control unit” or “control system” may be any computing device configured to send and/or receive information (e.g., including instructions) to/from various systems and/or devices. A control unit may comprise processing circuitry configured to execute operating routine(s) stored in a memory. The control unit may comprise, for example, a processor, microcontroller, application-specific integrated circuit (ASIC), field-programmable gate array (FPGA), and the like, any other digital and/or analog components, as well as combinations of the foregoing, and may further comprise inputs and outputs for processing control instructions, control signals, drive signals, power signals, sensor signals, and the like. All such computing devices and environments are intended to fall within the meaning of the term “controller,” “control unit,” “control system,” “processor,” or “processing circuitry” as used herein unless a different meaning is explicitly provided or otherwise clear from the context. The term “control unit” is not limited to a single device with a single processor, but may encompass multiple devices (e.g., computers) linked in a system, devices with multiple processors, special purpose devices, devices with various peripherals and input and output devices, software acting as a computer or server, and combinations of the above. In some implementations, the control unit may be configured to implement cloud processing, for example by invoking a remote processor.

Moreover, as used herein, the term “processor” may include one or more individual electronic processors, each of which may include one or more processing cores, and/or one or more programmable hardware elements. The processor may be or include any type of electronic processing device, including but not limited to central processing units (CPUs), graphics processing units (GPUs), ASICS, FPGAS, microcontrollers, digital signal processors (DSPs), or other devices capable of executing software instructions. When a device is referred to as “including a processor,” one or all of the individual electronic processors may be external to the device (e.g., to implement cloud or distributed computing). In implementations where a device has multiple processors and/or multiple processing cores, individual operations described herein may be performed by any one or more of the microprocessors or processing cores, in series or parallel, in any combination.

As used herein, the term “memory” may be any storage medium, including a non-volatile medium, e.g., a magnetic media or hard disk, optical storage, or flash memory; a volatile medium, such as system memory, e.g., random access memory (RAM) such as dynamic RAM (DRAM), synchronous dynamic RAM (SDRAM), static RAM (SRAM), extended data out (EDO) DRAM, extreme data rate dynamic (XDR) RAM, double data rate (DDR) SDRAM, etc.; on-chip memory; and/or an installation medium where appropriate, such as software media, e.g., a CD-ROM, or floppy disks, on which programs may be stored and/or data communications may be buffered. The term “memory” may also include other types of memory or combinations thereof. For the avoidance of doubt, cloud storage is contemplated in the definition of memory.

Before any aspects of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

The use of wearable sensors for full-body tracking is becoming increasingly important in areas such as healthcare, sports, rehabilitation, virtual reality (including augmented reality (AR) and mixed reality (MR)), human-computer interaction, robotics control, video gaming, telecommunications (e.g., interactions in the metaverse), special training for dangerous applications (e.g., bomb squad training), professional training (e.g., DIY or mechanical training), skills training (e.g., virtual plano lessons), biomechanical study and medical applications (e.g., rehabilitation), and so forth. Wearable sensors are portable and can conveniently be used in everyday environments, for example, outside of specialized laboratories. Body tracking using wearable sensors has a wide range of potential applications. For example, such full-body tracking systems can provide personalized feedback for improving physical performance or recovery, enhancing immersion experiences in virtual and augmented environments, and many others.

One significant challenge in full-body motion tracking lies in the wide range of motion scales across the body. For example, the scale and type of motion of a hand are very different than that of a limb. In particular, the motions of human torso, arms, and legs have a large range but fewer degrees of freedom. In contrast, the motions of human hands and fingers have a small range, but they are complicated with rich degrees of freedom. These differences make it challenging to track both types of motions in one system, especially when detailed hand tracking is unavailable.

The present disclosure describes systems and methods that can provide wearable full-body motion tracking, including detailed hand tracking. Such body tracking can be described as position data, which may be referred to as motion data, pose data, or configuration data. Position data can be described by degrees of freedom associated with the anatomically relevant degrees of motion. For example, position data of the hand may include defining many degrees of freedom (DOFs), including metacarpophalangeal (MCP) joint angle of the index finger, proximal interphalangeal (PIP) joint angle of the index finger, and so forth. In one non-limiting example, 22 DOFs may be used. In other non-limiting examples, the hand may be described by 27 DOFs, including 4 DOFs for each of the four fingers, 5 DOFs for the thumb, and 6 DOFs for the wrist (3 for rotation and 3 for translation). In other non-limiting implementations, the position model of the hand may be described by fewer DOFs, considering the physiological interdependencies of some joints. In some implementations, the position model can be described by a reduced number of DOFs that is sufficient for the desired application. For example, when characterizing a pinching task, the positions of the middle finger, ring finger, and pinky finger can be ignored. As another example, position data of the forearm may include an angle of the elbow's extension.

The position data may be distinguished as hand position data and body or limb position data. The hand position data, which may be referred to as hand configurations or hand configuration data, can be determined based on ultrasound data, as will be described. In some implementations, such hand configurations can be described by a desired number of degrees of freedom that describe the position of each finger (e.g., each phalange) and palm. The body and limb position data, which may be referred to as body and limb configurations or body and limb configuration data, can be determined based on IMU data, as will be described. In some implementations, such body and limb configurations can be described by 6 degrees of freedom for each body part (e.g., lower arm, upper arm), including 3 translational degrees of freedom (e.g., translation along x, y, and z) and three rotational degrees of freedom (e.g., rotation around x, y, and z).

The integrated system utilizes ultrasound sensors that provide detailed tracking of fine, small range motion, like that of the hands, combined with inertial measurement unit (IMU) sensors that provide tracking of large range motion, like that of the limbs and torso. The combination of ultrasound sensors and IMU sensors provides motion tracking over a wide range of motion scales and number of degrees of freedom. In this way, the described system can provide full-body motion tracking that can be used for a variety of applications, such as robotic motion training and task training, medical rehabilitation, biomechanics study, and so forth. Moreover, the tracking performance of the described systems and methods is not hindered from visual obstacles or other environmental variations.

In some implementations, the system further integrates one or more cameras or other sensors (e.g., other optical sensors, GPS receivers, electromagnetic sensors, electromyography (EMG) sensors, wireless tracking or indoor GPS receivers, radio frequency identification tags, infrared sensors, low-resolution ultrasound transducers, and so forth).

In some implementations, the described wearable hand tracking system (e.g., ultrasound or other imaging system) provides dexterous virtual hands, capable of accurately and continuously tracking the configurations and motions of individual fingers and palms in real-time during daily activities, may be used to increase the effectiveness the interactions among humans, virtual reality, and machines such as robots. However, widely adopted and easily accessible strategies for dexterous virtual hands are still unavailable using systems and methods according to comparative examples. Camera-based systems are used in comparative examples for hand tracking, particularly due to their compatibility with VR headsets. Additionally, quantitatively evaluating the performance of these tracking systems can be challenging because they are highly susceptible to uncontrollable factors, such as the viewing angle, visual obstacles, environmental light, and the like, and these systems only provide a limited field of view. These inherent issues limit the overall performance of these systems.

In some implementations, hand tracking is provided by continuous high-resolution imaging of a human hand's wrist assisted by an AI algorithm that can accurately and continuously track relevant DOFs of the hand's relevant fingers, palm, or both in real-time. Alternative hand tracking methods only recognize a limited number of predefined discrete hand gestures. In contrast, the wearable high-resolution ultrasound wristband according to the present disclosure can continuously (e.g., with precision of at least 5° and 1 mm) and quantitatively track many DOFs (e.g., 22 or more DOFs) of the hand's five fingers and palm.

As a non-limiting example, the system can track 22 DOFs that describe all five fingers and the palm in real-time. As a non-limiting example, continuous tracking can refer to the continuous description (e.g., angle or translational position) of each DOF. In this way, the system does not require a discrete set of pre-defined positions but instead allows for an arbitrary description of the position characterized by a continuous rotational angle (e.g., 0°-360°) or translation (e.g., 0-2 m) of each DOF (e.g., joint, overall hand or arm position). In some implementations, the precision of the continuous tracking may be determined by the precision required for the application or anatomical region. As non-limiting examples, the continuous tracking can provide precision of <10°, <5°, <3°, <2°, <1°, 1°-5°, or 2°-5°. As a non-limiting example, the continuous tracking can provide precision of 2°-5° for each DOF of the hand and at least 3° of the upper arm. The continuous tracking can also provide a continuous translational position (e.g., in x, y, and z). As non-limiting examples, the translational position can be described with precision of <0.1 mm, <1 mm, <5 mm, <10 mm, <50 mm, and so forth.

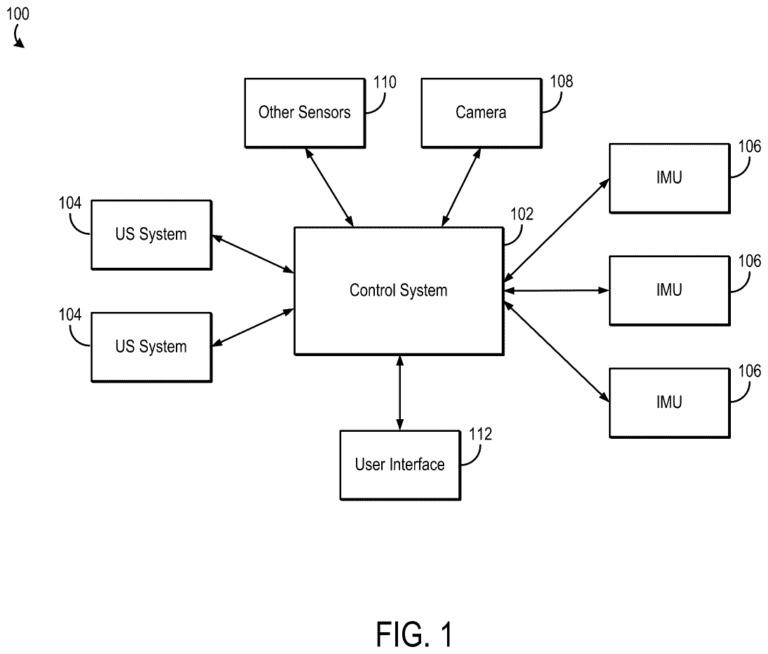

Referring now to FIG. 1, a body-tracking system 100 is presented. The system 100 includes a central control system 102 in communication with one or more wearable sensors or other data inputs. The communication between the sensors and control system 102 can be wired or wireless (e.g., Bluetooth, Wi-Fi, and so forth). The control system 102 may be located on a virtual reality headset, mounted on one of the wearable sensors, coupled to or worn on the wearer (e.g., in a fanny pack), or external to the wearable sensors (e.g., in a server room).

In general, the system 100 includes one or more wearable ultrasound (US) systems 104 and one or more wearable inertial measurement unit (IMU) systems 106. In some implementations, the ultrasound systems 104 may include a wearable wireless ultrasound system, as described in PCT Patent Publication No. WO2025/174729A1, which is incorporated herein by reference in its entirety and further described below. While the examples provided in the present disclosure are written based on high resolution ultrasound, the present disclosure is not so limited. In practice, the present disclosure may be implemented using any imaging modality that is capable of accurately imaging the interior structure of the wrist. In some examples, the present disclosure may be practiced in implementations using photoacoustic imaging, MRI, optical computed tomography (OCT) imaging, and/or infrared (IR) imaging so long as such implementations have sufficient imaging resolution, imaging depth, device size, etc. to operate in accordance with the methods set forth herein. In this way, the ultrasound system 104 may be replaced by another imaging system, such as MRI system, OCT system, IR system, and so forth.

The ultrasound system 104 may be referred to as an ultrasound probe, imaging probe, or ultrasound sensor. The ultrasound system 104 can provide continuous high-resolution imaging of a body structure. For example, the body structure may be a human hand, including all five fingers and palm. The ultrasound system 104 may have the form factor of a wristband that can be worn on a wrist of a subject. In some implementations, the system 100 includes two ultrasound wristbands, one worn on each wrist. Such system can provide detailed tracking of hand and finger motion of one or both hands.

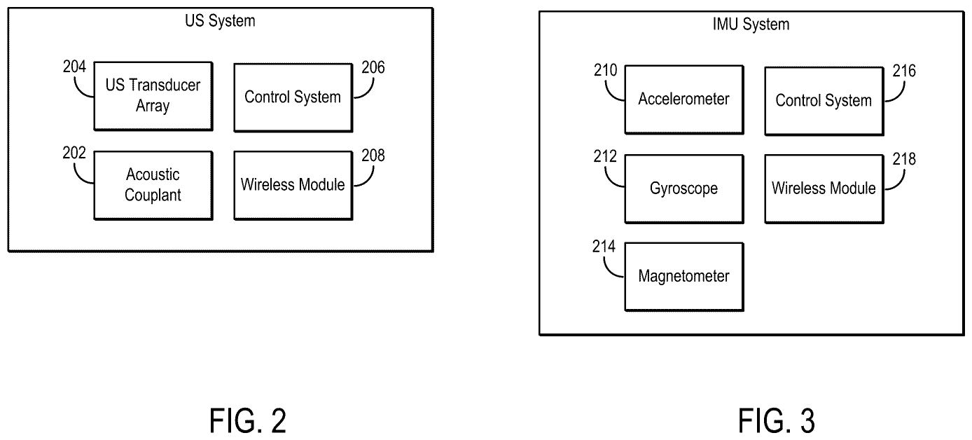

As shown in FIG. 2, the ultrasound system 104 may include an acoustic couplant 202, an ultrasound transducer array 204, a control system 206, and a wireless module 208. In some implementations, the acoustic couplant 202 may be an adhesive couplant, including an adhesive and hydrogel or elastomers. In some implementations, the acoustic couplant 202 may be a non-adhesive couplant based on a hydrogel, polymer, or elastomers. In other implementations the acoustic couplant 202 may be a non-adhesive couplant that includes elastomers and greases. Further details and examples of the couplant are provided below and in PCT Publication No. WO2025/174729A1, the entire contents of which is incorporated herein by reference. The ultrasound transducer array 204 may include several (e.g., 256) piezoelectric components configured to measure ultrasound echo data that is processed by the ultrasound system 104 to provide imaging or ultrasound data.

The control system 206 provides electronic control of the ultrasound system 104. In some configurations, the control system 206 can include a signal processing system to provide local data processing. In this way, the control system 206 can receive the ultrasound data measured by the US transducer array 204 to process the ultrasound data. Such processing may include cleaning, filtering, or transforming the data to provide pre-processed data, which may include imaging data, that can be transferred to the central control system 102 to be further processed or analyzed. Processing may additionally include analyzing the ultrasound imaging data to generate position data (e.g., tracked hand configurations). Analyzing the ultrasound data to generate position data my include processing the imaging data using machine learning computing to determine a quantified parameter (e.g., angle or translation) of each of the defined degrees of freedom. The generated position data can be transferred to the central control system 102 for integration with the whole system 100 in order to characterize a position of an extended body region (e.g., whole arm, whole body).

In other configurations, the raw ultrasound data measured by the ultrasound transducer array 204 or ultrasound imaging data can be transmitted to the central control system 102 for processing or analyzing. Such communication can be facilitated using the wireless module 208 that provides two-way communication between the ultrasound system 104 and the central control system 102 or other parts of the tracking system 100. The ultrasound signal can be analyzed using the central control system 102, which may advantageously provide stronger processing capability than that of the local control system 206 of the ultrasound system 104. Such processing can be performed using machine learning algorithms to produce position data, such as hand configuration. Several different machine learning algorithms, such as a convolutional neural network, may be used; further details of example machine learning algorithms are provided below and in PCT Publication No. WO2025/174729, which is incorporated herein by reference.

Referring again to FIG. 1, the tracking system 100 can also include one or more inertial measurement unit (IMU) systems 106. Such IMU systems can be wearable and configured to be attached to the forearm, upper arm, lower body, or other body structure, to track the motion of other body parts. As one non-limiting example, the IMU systems 106 may include commercially available IMU sensors, such as a Bosch BMI160.

The IMU systems 106 include sensors (e.g., accelerometer, gyroscope, magnetometer) that provide inertia data, which may include acceleration data, rotation data, and magnetic field data. As shown in FIG. 2, each IMU system includes one or more accelerometers 210 to provide acceleration data and one or more gyroscopes 212 to provide rotation data. Each IMU system may also optionally include one or more magnetometers that provide magnetic field data. The acceleration data, rotation data, and optional magnetic field data can be combined to provide inertia data that can be analyzed locally by the control system 216 or transmitted to the central control system 102 via the wireless module 218 for further processing. For example, the inertia data may be used by an algorithm, such as a machine learning algorithm (e.g., convolutional neural network), to classify position or motion of the associated body structure. In this way, the inertia data can be processed by a control system (e.g., 102 or 216) to provide motion or position data, including position and orientation of the limbs (e.g., upper arm, forearm, thigh, calf), torso, waist, hips, neck, head, and other body parts. Such position data may be defined based on a movement measured relative to the previous position measured.

Referring again to FIG. 1, the tracking system 100 may further include one or more external cameras 108 or other optical sensors (e.g., depth sensors). The optical data can be transferred wirelessly to the central control system 102 to help generate position data along with the inertia data. Similarly, the system 100 may include additional sensors 110 that provide sensor data to the control system 102 that can be used in conjunction with the inertia data to generate position data. Such sensors may include other optical sensors, GPS receivers, electromyography (EMG) sensors, electromagnetic sensors wireless tracking or indoor GPS receivers, radio frequency identification tags, infrared sensors, ultrasound transducers, low resolution ultrasound transducers, electrical impedance sensors, and so forth. In some implementations, the cameras 108 or other sensors 110 can be used to calibrate the inertia data. For example, the IMUs 106 can be used for continuous tracking of full body motion, while the cameras 108 can be used to periodically calibrate the inertia data with externally measured position data. In some configurations, the cameras 108 can be used to correct erroneous position data generated from inertia data if camera data is available. In other configurations, the cameras 108 can be used to calibrate the position data in space (e.g., appropriately place the position data with respect to the subject's surroundings or with respect to another subject). In other configurations, the cameras 108 can be included and used to provide ground truth data in the training of a machine learning model and omitted when applying the machine learning model based on US and IMU sensors.

In some implementations, the system 100 may also include a user interface 112 that can transmit other data or user inputs to the control system 102. For example, the user interface 112 may be used to assign each sensor used to a corresponding body part (e.g., US sensor 1 on right wrist, US sensor 2 on left wrist, IMU 1 on right forearm, IMU 2 on left forearm, IMU 3 on right upper arm, and so forth) or input subject data (e.g., age, anatomical measurements of the subject). The user interface 112 can also provide additional user control (e.g., calibration, adjusting system parameters, and so forth).

The control system 102 is configured to combine all of the data measured from the ultrasound systems 104, IMUs 106, optional cameras 108, and optional additional sensors 110 to produce integrated position data. This integrated position data advantageously characterizes body movement on varying scales, including the small and complex movement of the hands and the larger, more restricted movement of the limbs and trunk. In this way, the control system 102 can generate continuous full-body position data of the subject by integrating ultrasound data and inertia data. For example, the position data generated using the ultrasound systems worn on the wrists can be integrated with the position data generated using the IMUs worn on the forearms.

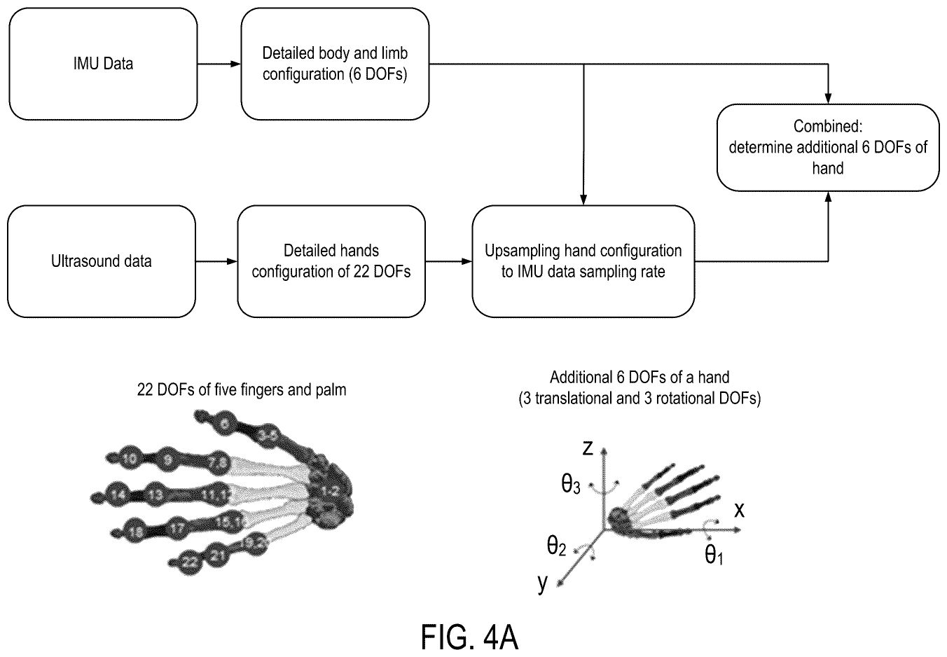

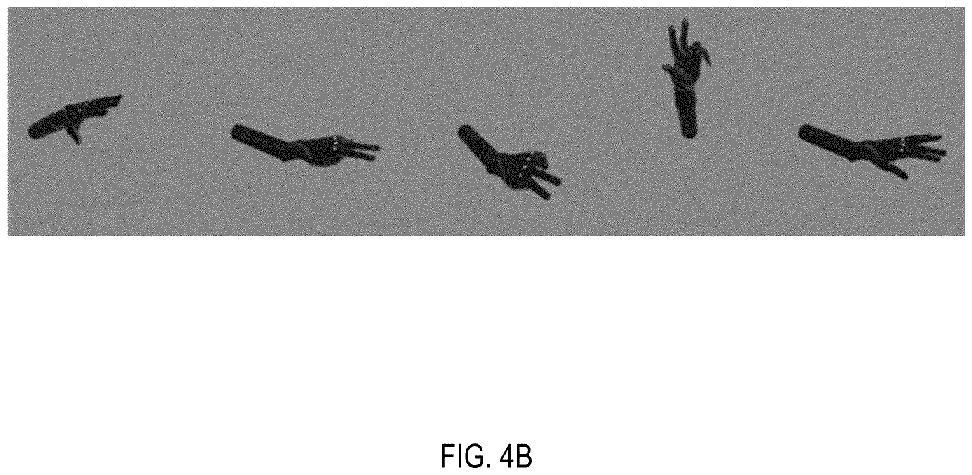

Integrating the data can include mapping the data to combine the position data in space and time. Data can be integrated based on the time synchronization, as shown in FIG. 4A. In some implementations, the IMU data and ultrasound data can be separately analyzed in order produce detailed body and limb configurations and detailed hand configurations, respectively. As one non-limiting example, the hand configurations may be described by 22 degrees of freedom that describe the positions of the fingers and palm (see FIG. 4A), while the body and limb configurations can be described by 6 degrees of freedom for each limb (e.g., 3 translational and 3 rotational). The body and hand configurations can be matched in temporal sampling. For example, the hand configuration data can be upsampled to match the sampling rate of the IMU data. The body and hand configurations can then be combined, which provides 6 additional degrees of freedom for the hand configurations that describe the translation and rotation of the whole hand position. In this way, the hand configuration data and the body/limb configuration data can be integrated in time and space.

FIG. 4B shows an example of integrated data for a subject's hand and forearm. The detailed position data of the hand (as generated by an ultrasound wristband system) was integrated with the forearm position, as measured from an IMU. This provides an integrated position model of the full forearm and hand, combining the larger motion of the arm with the fine detailed motion of the fingers and hand. This integration can also be performed for a full body or another portion of a full body (e.g., whole upper body, whole right arm).

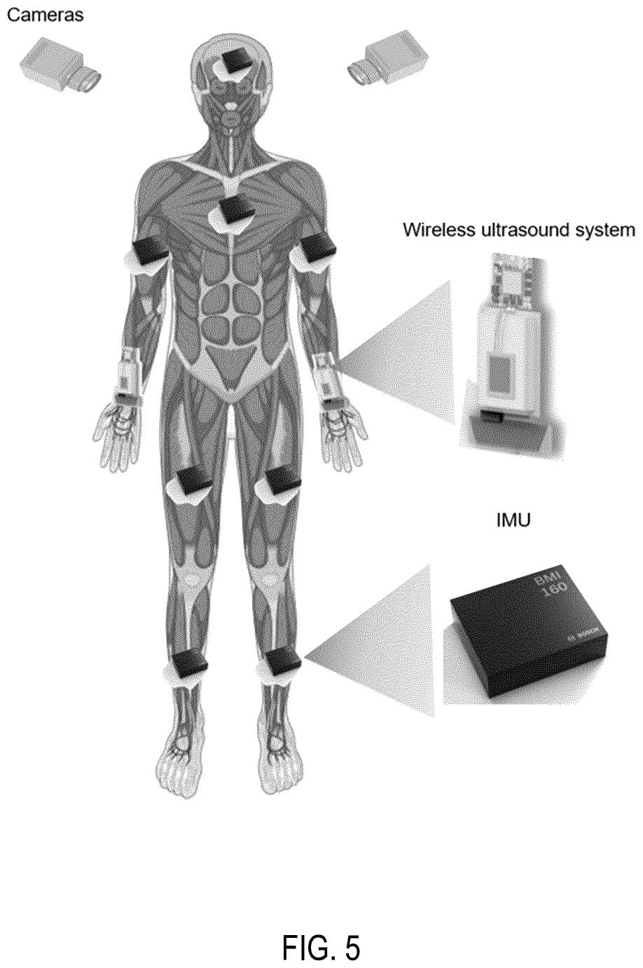

FIG. 5 shows an illustration of one non-limiting example system. In this implementation, the system includes eight IMUs. The IMUs are distributed on the body of the subject, including one on the head, one on the chest, one on each upper arm, one on each upper leg, and one on each lower leg. The system further includes two ultrasound wristband systems, one worn on each wrist. The system also includes two external cameras. This example system can provide position data for the full body using the IMUs and ultrasound wristbands, while calibrating the system using the two external cameras.

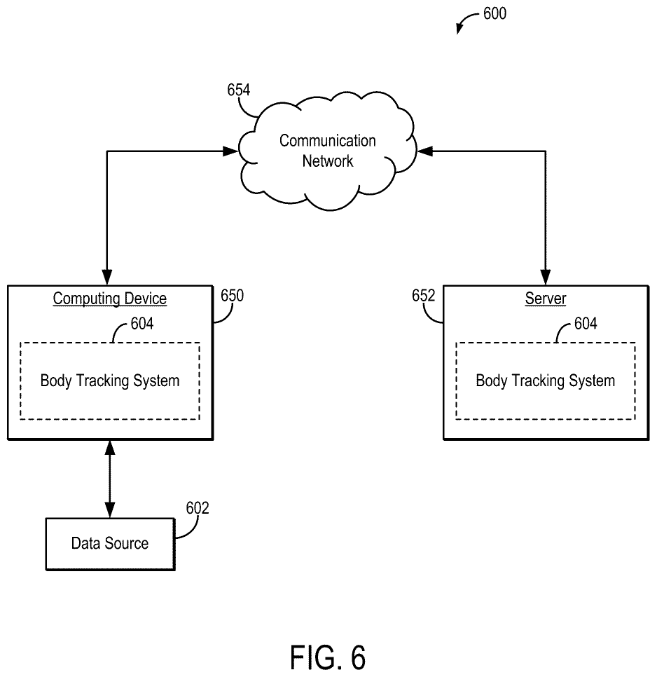

Referring now to FIG. 6, an example of a tracking control system 600 is shown, which may be used in accordance with some aspects of the systems and methods described in the present disclosure. As shown in FIG. 6, a computing device 650 can receive one or more types of data (e.g., sensor data, inertia data, acceleration data, rotational data, ultrasound data) from data source 602. In some configurations, computing device 650 can execute at least a portion of a tracking system 604 to measure the data. In some configurations, the tracking system 604 can implement an automated pipeline to provide labeled position data or pre-processed data.

Additionally or alternatively, in some configurations, the computing device 650 can communicate information about data received from the data source 602 to a server 652 over a communication network 654, which can execute at least a portion of the tracking system 604. In such configurations, the server 652 can return information to the computing device 650 (and/or any other suitable computing device) indicative of an output of the tracking system 604.

In some configurations, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also process sensor data.

In some configurations, data source 602 can be any suitable source of data (e.g., measurement or sensor data, pre-processed data, generated position data, camera images or videos), such as an ultrasound system, IMU system, camera, another computing device (e.g., a server storing measurement or sensor data, pre-processed data, generated position data, camera images or videos, calibration data), and so on. In some configurations, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some configurations, data source 602 can be located locally and/or remotely from computing device 650, and can communicate data to computing device 650 (and/or server 652) via a communication network (e.g., communication network 654).

In some configurations, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

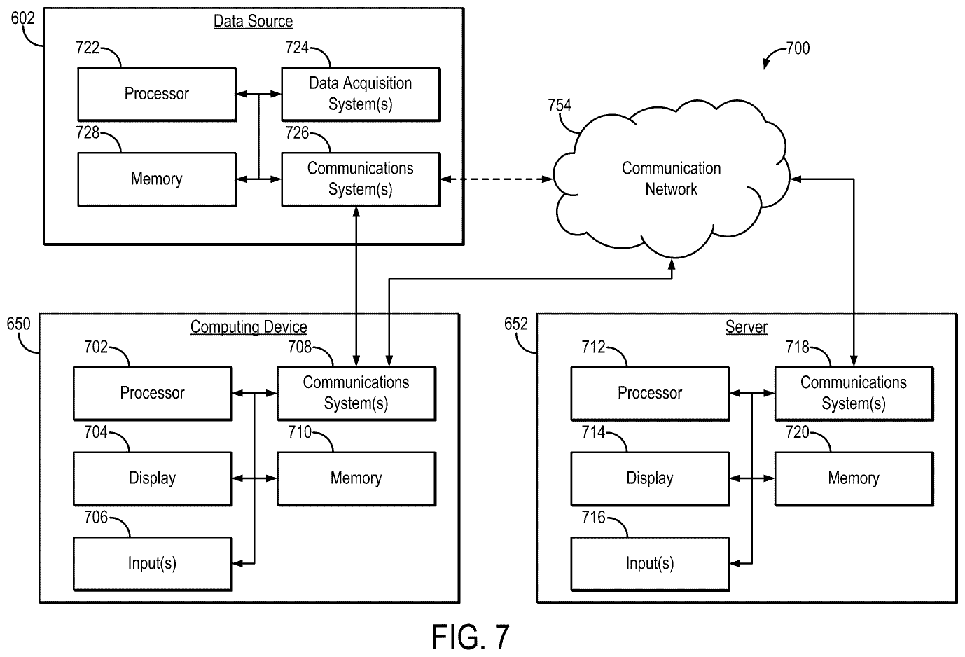

Referring now to FIG. 7, an example of hardware 700 that can be used to implement data source 602, computing device 650, and server 652 in accordance with some configurations of the systems and methods described in the present disclosure is shown.

As shown in FIG. 7, in some configurations, computing device 650 can include a processor 702, a display 704, one or more inputs 706, one or more communication systems 708, and/or memory 710. In some configurations, processor 702 can be any suitable hardware processor or combination of processors, such as a central processing unit (“CPU”), a graphics processing unit (“GPU”), and so on. In some configurations, display 704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 706 can include any suitable input devices and/or sensors that can be used to receive user input, such as a keyboard, a mouse, a touchscreen, a microphone, and so on.

In some configurations, communications systems 708 can include any suitable hardware, firmware, and/or software for communicating information over communication network 754 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some configurations, memory 710 can include any suitable storage device or devices that can be used to store instructions, values, data, or the like, that can be used, for example, by processor 702 to present content using display 704, to communicate with server 752 via communications system(s) 708, and so on. Memory 710 can include any suitable volatile memory, non-volatile memory, storage, or any suitable combination thereof. For example, memory 710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 710 can have encoded thereon, or otherwise stored therein, a computer program for controlling operation of computing device 750. In such configurations, processor 702 can execute at least a portion of the computer program to present content (e.g., images, user interfaces, graphics, tables), receive content from server 752, transmit information to server 752, and so on. For example, the processor 702 and the memory 710 can be configured to perform the methods described herein.

In some configurations, server 752 can include a processor 712, a display 714, one or more inputs 716, one or more communications systems 718, and/or memory 720. In some configurations, processor 712 can be any suitable hardware processor or combination of processors, such as a CPU, a GPU, and so on. In some configurations, display 714 can include any suitable display devices, such as an LCD screen, LED display, OLED display, electrophoretic display, a computer monitor, a touchscreen, a television, and so on. In some configurations, inputs 716 can include any suitable input devices and/or sensors that can be used to receive user input, such as a keyboard, a mouse, a touchscreen, a microphone, and so on.

In some configurations, communications systems 718 can include any suitable hardware, firmware, and/or software for communicating information over communication network 754 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some configurations, memory 720 can include any suitable storage device or devices that can be used to store instructions, values, data, or the like, that can be used, for example, by processor 712 to present content using display 714, to communicate with one or more computing devices 750, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 720 can have encoded thereon a server program for controlling operation of server 752. In such configurations, processor 712 can execute at least a portion of the server program to transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone), and so on.

In some configurations, the server 752 is configured to perform the methods described in the present disclosure. For example, the processor 712 and memory 720 can be configured to perform the methods described herein.

In some configurations, data source 702 can include a processor 722, one or more data acquisition systems 724, one or more communications systems 726, and/or memory 728. In some configurations, processor 722 can be any suitable hardware processor or combination of processors, such as a CPU, a GPU, and so on. In some configurations, the one or more data acquisition systems 724 are generally configured to acquire sensor data, images, or both, and can include cameras, ultrasound systems, IMUs, or other sensors. Additionally or alternatively, in some configurations, the one or more data acquisition systems 724 can include any suitable hardware, firmware, and/or software for coupling to and/or controlling operations of the sensor systems. In some configurations, one or more portions of the data acquisition system(s) 724 can be removable and/or replaceable.

Note that, although not shown, data source 702 can include any suitable inputs and/or outputs. For example, data source 702 can include input devices and/or sensors that can be used to receive user input, such as a keyboard, a mouse, a touchscreen, a microphone, a trackpad, a trackball, and so on. As another example, data source 702 can include any suitable display devices, such as an LCD screen, an LED display, an OLED display, an electrophoretic display, a computer monitor, a touchscreen, a television, etc., one or more speakers, and so on.

In some configurations, communications systems 726 can include any suitable hardware, firmware, and/or software for communicating information to computing device 750 (and, in some configurations, over communication network 754 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate position data from the sensor data; present content (e.g., data, images, a user interface) using a display; communicate with one or more computing devices 750; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

### EXAMPLES

### Example Machine Learning Implementation

As previously described, one or more trained machine learning algorithms can be used to determine position data from sensor data measured from the US system, the IMU system, other sensors, a camera, or a combination thereof. In some implementations, the machine learning algorithm may be configured to separately determine the position data of two different anatomical regions (e.g., hand and arm). In this way, the machine learning algorithm can treat each sensor independently. The output position data for each region can then be integrated to describe the position of a larger anatomical region (e.g., the full arm, including the hand). In other implementations, the machine learning algorithm can jointly consider each sensor to jointly determine position data for multiple anatomical regions (e.g., arm and hand) to determine the position of a larger anatomical region (e.g., full extremity).

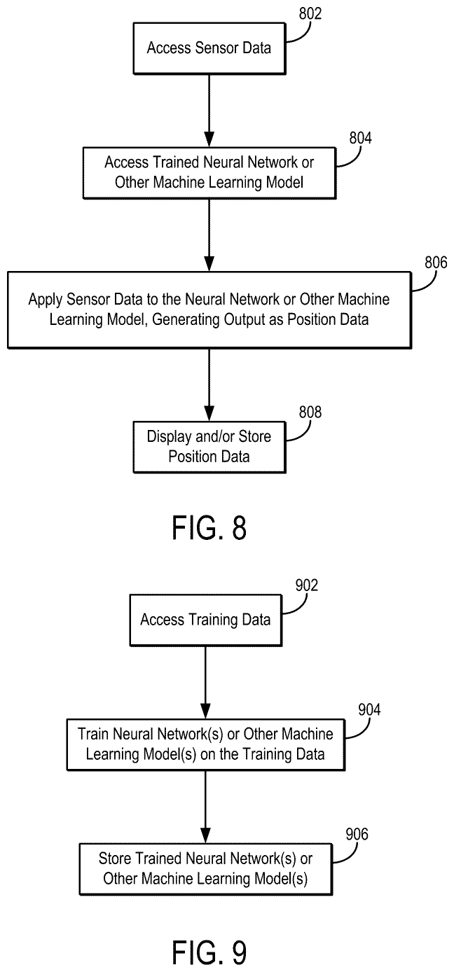

Referring now to FIG. 8, a flowchart is illustrated as setting forth the steps of an example method for generating classified feature data using a suitably trained neural network or other machine learning algorithm. As will be described, the neural network or other machine learning algorithm takes sensor data as input data and generates position data as output data. The sensor data may include ultrasound data measured from one or more ultrasound systems, IMU data measured from one or more IMU systems, other sensor data measured from other sensors, or a combination thereof. As an example, the position data can be indicative of a description of position of an anatomical body part (e.g., hand, each finger, forearm, upper arm, wrist, palm, and so forth). For example, position data can be described by degrees of freedom (e.g., angle or translation) associated with the anatomically relevant degrees of motion (e.g., 22 for the hand).

The method includes accessing sensor data with a computer system, as indicated at step 802. Accessing the sensor data may include retrieving such data from a memory or other suitable data storage device or medium. Additionally or alternatively, accessing the sensor data may include acquiring such data with a body-tracking system or part of a body-tracking system (e.g., US system, IMU system) and transferring or otherwise communicating the data to the computer system or control system, which may be a part of the body tracking system.

A trained neural network (or other suitable machine learning algorithm) is then accessed with the computer system, as indicated at step 804. Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

In general, the neural network is trained, or has been trained, on training data in order to determine position data for various anatomical regions (e.g., hand, arm) from sensor data measured from several types of sensors (e.g., ultrasound, IMU, camera, other sensors). An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs. In an example in which the artificial neural network determines position data of a hand, the output layer may include, for example, a number of different nodes, where each different node corresponds to a different degree of freedom. A first node may indicate an angle of a first joint, a second node may indicate an angle of a second joint, and so forth. In another example in which the artificial neural network determines position data of a hand and an arm, a first node may indicate a position of the hand and a second node may indicate a position of the arm.

The sensor data are then input to the one or more trained neural networks, generating output as position data, as indicated at step 806.

The position data generated by inputting the sensor data to the trained neural network(s) can then be displayed to a user, stored for later use or further processing, or both, as indicated at step 808.

Referring now to FIG. 9, a flowchart is illustrated as setting forth the steps of an example method for training one or more neural networks (or other suitable machine learning algorithms) on training data, such that the one or more neural networks are trained to receive sensor as input data in order to generate position data as output data, where the position data are indicative of a description of a parameter of a degree of freedom associated with an anatomical region (e.g., an angle of a joint or elbow, an angle or linear position of a wrist, and so forth).

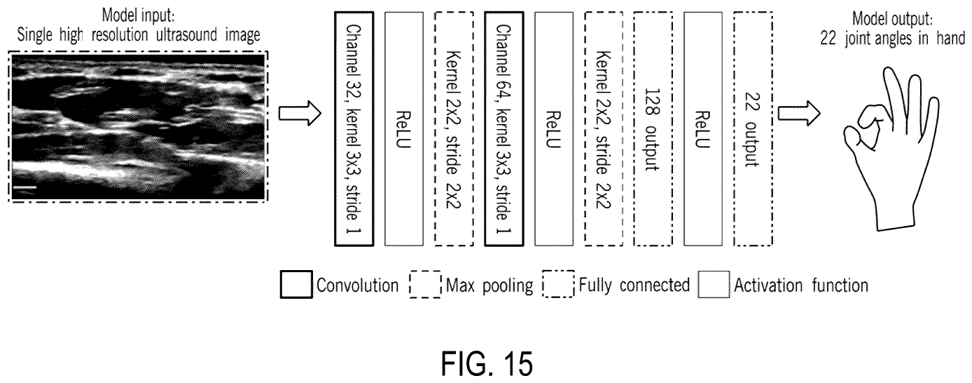

In general, the neural network(s) can implement any number of different neural network architectures. For instance, the neural network(s) could implement a convolutional neural network, a residual neural network, or the like. Alternatively, the neural network(s) could be replaced with other suitable machine learning or artificial intelligence algorithms, such as those based on supervised learning, unsupervised learning, deep learning, ensemble learning, dimensionality reduction, and so on. A non-limiting example network implementation is described below and illustrated in FIG. 15.

Referring again to FIG. 9, the method includes accessing training data with a computer system, as indicated at step 902. Accessing the training data may include retrieving such data from a memory or other suitable data storage device or medium. Alternatively, accessing the training data may include acquiring such data with a body-tracking system and transferring or otherwise communicating the data to the computer system.

In general, the training data can include sensor data, which may include US data, IMU data, other sensor data, or a combination thereof. Additionally, the training data may include other data, such as camera data, that can be used as ground truth data. In some embodiments, the training data may include sensor data that have been labeled (e.g., labeled with a corresponding position determined using a camera or other ground truth source).

The method can include assembling training data from a body-tracking system using a computer system. This step may include assembling the sensor data into an appropriate data structure on which the neural network or other machine learning algorithm can be trained.

One or more neural networks (or other suitable machine learning algorithms) are trained on the training data, as indicated at step 904. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as position data. The artificial neural network then compares the generated output with the actual output of the training example in order to evaluate the quality of the position data. For instance, the position data can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., positions). In these instances, the artificial neural network is configured to learn a general rule or model that maps the inputs to the outputs based on the provided example input-output pairs.

The one or more trained neural networks are then stored for later use, as indicated at step 906. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

### Example Body Tracking System

The following example provides further details of example body tracking systems described in the present disclosure.

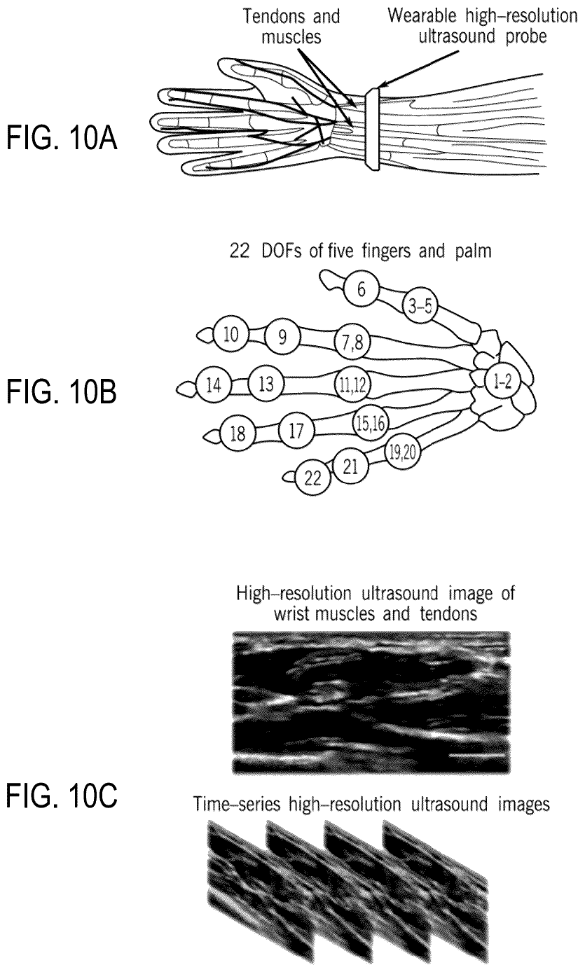

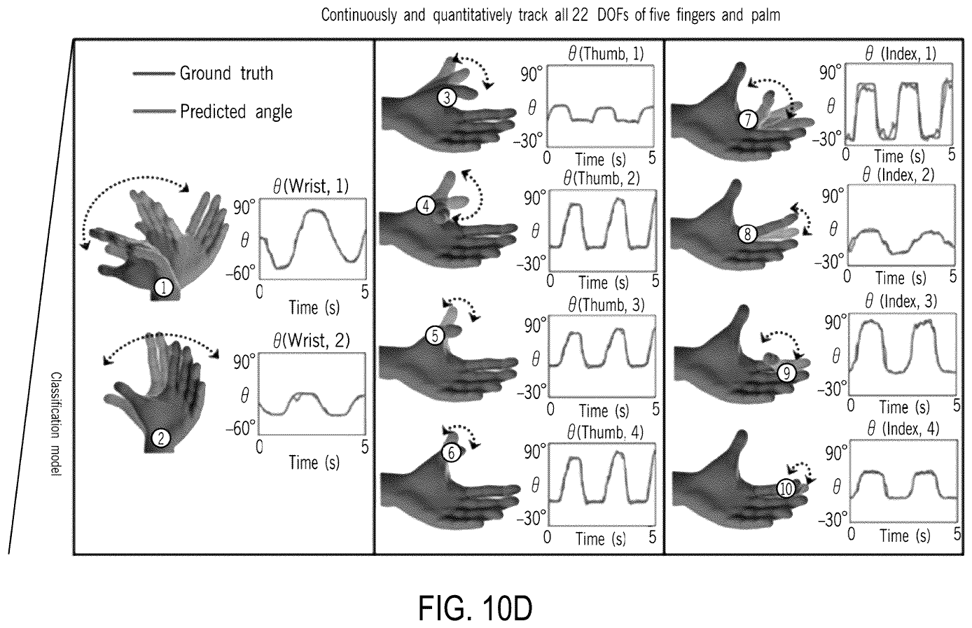

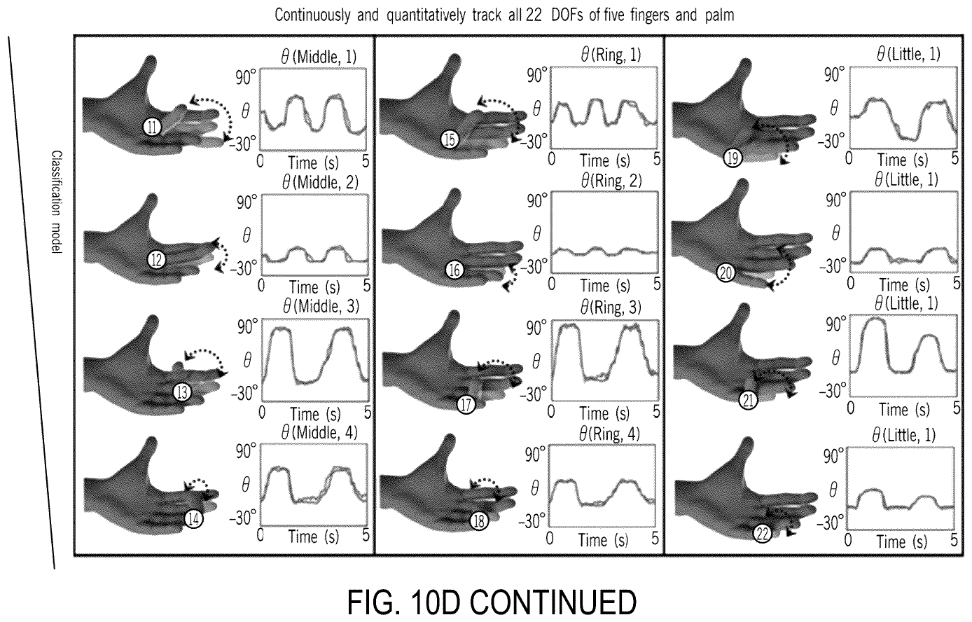

FIGS. 10A-10D illustrate a non-limiting example implementation of the US system. As shown in FIG. 10A, the wearable high-resolution ultrasound probe is stably placed on the wrist to capture images of the tendons and muscles through time. FIG. 10B shows schematics of the 22 DOFs of the hand's five fingers and palm. FIG. 10C shows a time-series of high-resolution ultrasound images of the wrist tendon-muscle anatomy as acquired by the ultrasound wristband. The scale bar is 1 cm. As illustrated in FIG. 10D, the ultrasound images are processed by a regression machine learning model to continuously quantify the 22 DOFs of the hand in real-time.

As a non-limiting example, the regression machine learning model can provide an output of 22 DOFs to describe the hand, 6 DOF to describe the lower arm, and 6 DOF to describe the upper arm. The machine learning algorithm advantageously processes the sensor data quickly to provide real-time feedback; processing the sensor data is advantageously not the limiting factor of the delay. In some implementations, the latency of the position output is limited by the time it takes to acquire ultrasound images and transmit (e.g., wirelessly) the ultrasound data to the control system for processing. In some implementations, the frame rate of the ultrasound imaging can be 30 Hz, 60-120 Hz, or even 500 Hz or more. Thus, in some implementations, the time for data transmission may be the limiting factor in the overall latency. As non-limiting examples, the real-time position output can be provided with a delay <1 s, <0.5 s, <100 ms, <50 ms, <25 ms, <20 ms, <10 ms, or <1 ms.

The present disclosure may be embodied as a high-resolution ultrasound wristband, integrated with an AI algorithm, that can continuously track all 22 DOFs of a human hand's five fingers and palm in real-time during daily activities with high tracking accuracy (<1.5° root mean square error) and low processing latency (<1 ms). The AI algorithm may be included in the wearable device itself or may be stored on an external device (e.g., on a VR headset of the wearer). In implementations where the algorithm is external to the wristband, the wristband may be configured to communicate data with the external device. The high-resolution ultrasound wristband enables previously inaccessible and important applications, including camera-free and hand-free tracking of various continuous and subtle hand motions for intuitive and versatile controls of virtual reality and robotic hands. The performances of the ultra-dexterous virtual hands are superior to those of all comparative virtual hands based on camera-free hand-free armbands. Table 1 is a comparison of comparative example devices (EMG and low-resolution ultrasound) with devices according to the present disclosure.

TABLE 1

| | Device type | | | | |
|---|---|---|---|---|---|
| | | Low-resolution | | High-resolution | |
| | EMG | ultrasound | | ultrasound | |
| Wearing position | Forearm | Forearm | | Wrist | |
| # of discrete | 4-21 | 12-15 | | Any | |
| gestures | | | | | |
| # of continuous | N/A | 0-5 | | 22 | |
| DOFs | | | | | |
| Latency | 20-250 ms | 30-279 | ms | <1 | ms |
| Frequency | N/A | 1-9.5 | MHz | 18 | MHz |

| | | | |
|---|---|---|---|
| Number of sensors | 4-128 | 8-128 | 256 |

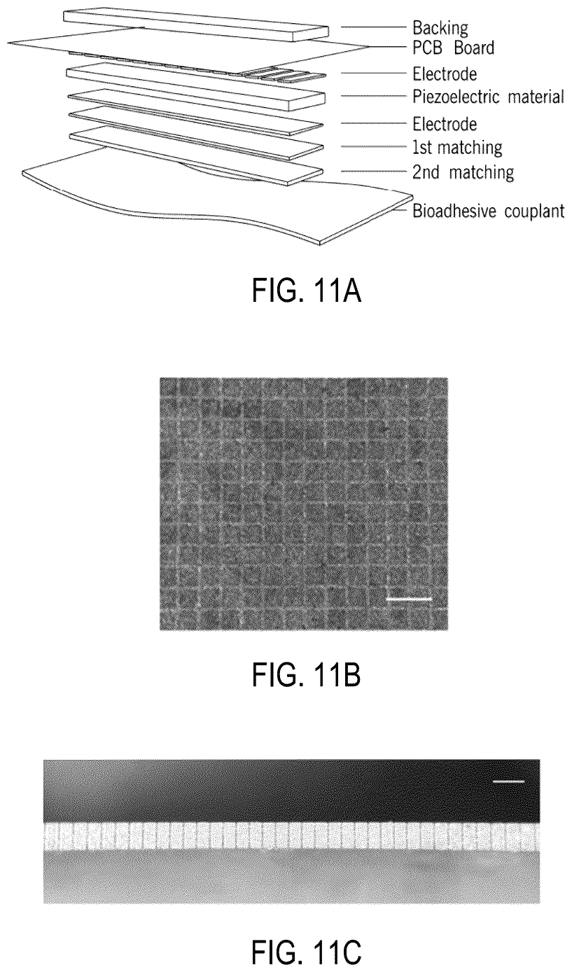

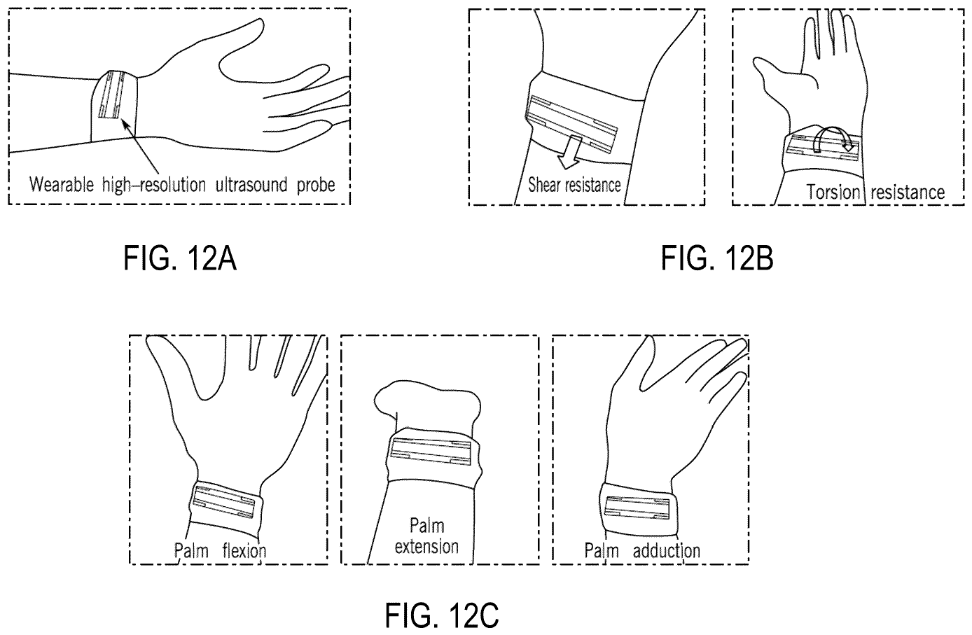

In one example, the wristband of the present disclosure comprises a high-resolution wearable ultrasound probe, as illustrated in FIGS. 11A-11C, and an ultrasound couplant, as illustrated in FIGS. 12A-12C. The ultrasound couplant can be made of water, water-based gel, hydrogel, oil, oil-based gel, Glycerin, etc. and other compound materials or layered structures. FIG. 11A shows the layered ultrasound probe. An example of design specifications is shown in Table 2. FIG. 11B shows a photo of 1-3 composite piezoelectric material from the top view. FIG. 11C shows a photo of 1-3 composite lead zirconate titanate (PZT)-5 from the side view. Scale bars indicate 100 μm. As shown in FIG. 11A, an example of a wearable imaging probe comprises a backing layer; a circuit layer; a piezoelectric layer including a first electrode, a piezoelectric material, and a second electrode; a first matching layer; and a second matching layer. In some examples, the circuit layer may include a processor and a memory, but in other examples the memory may be disposed on an external device (e.g., a VR headset of the wearer).

While FIGS. 11A-11C show one example implementation of a wearable ultrasound probe in accordance with the present disclosure, other implementations of a wearable ultrasound probe in accordance with the present disclosure may implement a piezoelectric micromachine ultrasound transducer (PMUT), a capacitive micromachined ultrasound transducer (CMUT), and the like. Moreover, while certain examples of the wearable ultrasound probe described herein provide information regarding 22 DOFs of the hand, the ultrasound probe may be combined with or complemented by another sensing modality (e.g., an optical tracking system, an inertial measurement unit (IMU), and the like) to realize full hand motion tracking, including information regarding 27 DOFs of freedom of the hand.

FIG. 12A shows a photo of the wearable high-resolution ultrasound probe of FIGS. 11A-11C imaging the wrist. As illustrated in FIG. 12B, the couplant may include hydrogel, an elastic solid, which is resistant to shear and torsion deformation. As shown FIG. 12C, the hydrogel maintains its shape during large-scale hand motions, ensuring a stable ultrasound imaging window.

TABLE 2
Specifications

| Piezoelectric | 1-3 composite PZT-5 | |
|---|---|---|
| Center frequency | 18 | MHz |
| Element pitch | 0.2 | mm |
| Kerf | 20 | μm |
| Azimuthal length | 51.2 | mm |
| Elevational width | 4.0 | mm |
| Elevational focal depth | 20 | mm |
| Backing | E-solder 3022 | |
| First matching layer | 2-3 μm silver\epoxy composite | |
| Second matching layer | Parylene C | |
| Zₘ₁ | 6.1 MRayl (33 μm) | |
| Vₘ₁ | 1961 | m/s |
| Zₘ₂ | 2.5 MRayl (35 μm) | |
| Vₘ₂ | 1900 | m/s |
| Z_{backing} | 5.9 | MRayl |
| V_{backing} | 2080 | m/s |
| Backing attenuation | 3.67 | dB/mm/MHz |

Thus, in one example, a high-resolution ultrasound probe in accordance with the present disclosure comprises an array of 256 high-performance piezoelectric components with a center frequency of 18 MHz. In this example, the ultrasound probe has an overall size of 5.5 cm in length, 4 mm in width, 5 mm in thickness, and a weight of 15 g. The ultrasound probe is connected to a control unit (e.g., Verasonics Vantage system) with two flexible flat cables. The ultrasound probe provides a field of view measuring 5.2 cm in width and 3 cm in depth, covering the muscles and tendons in the wrist that control five fingers and the palm. The axial resolution of the wristband is 0.67 mm, and the lateral resolution is 1.86 mm at 4 cm depth.

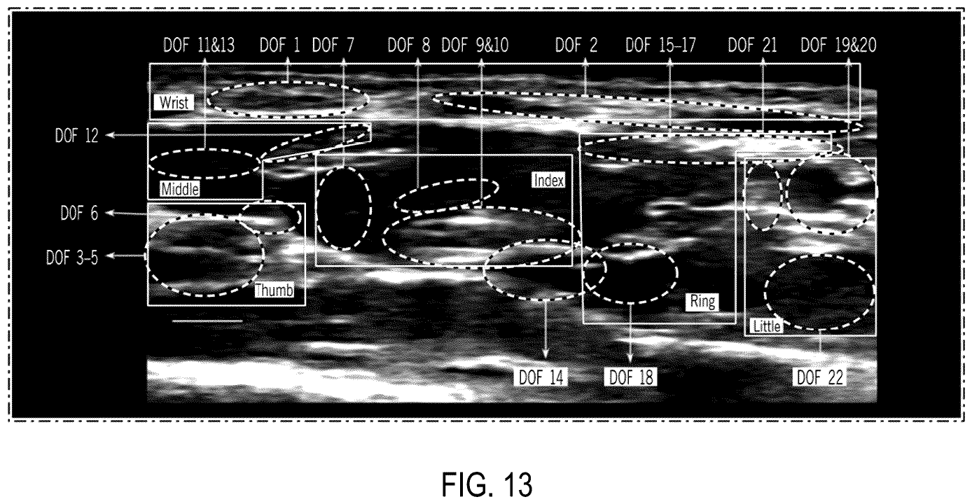

In one example, during use the wristband is positioned 2 cm away from the carpus and perpendicular to the ulna. The wristband provides continuous high-resolution imaging of the muscles and tendons in the wrist at a frequency of 30 images per second. On each image, six regions characterize the configurations of the five fingers and the palm, respectively, as shown in FIG. 13. In particular, as shown in FIG. 13, on the high-resolution ultrasound image of the wrist, six regions characterize the configurations of the five fingers and the palm, respectively, indicated by rectangles. Sub-regions in these six regions further characterize the DOFs of individual joints in the corresponding fingers or palm, indicated by green circles. The scale bar is 5 mm.

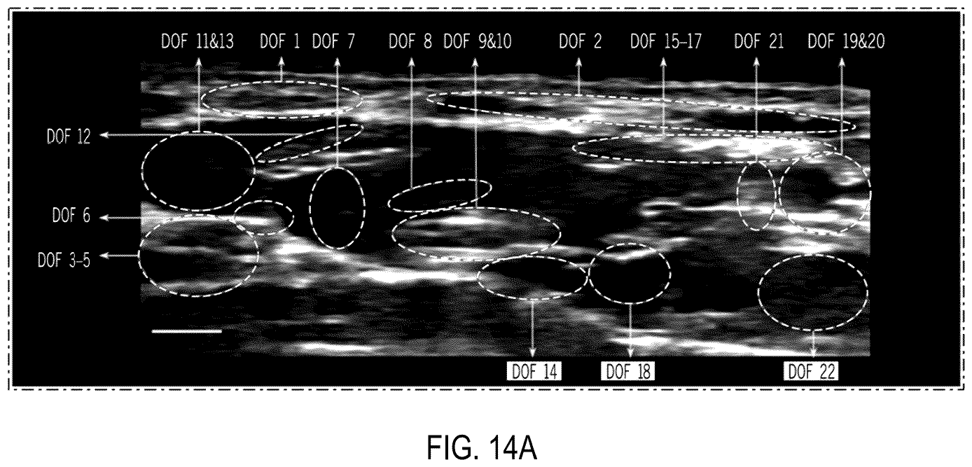

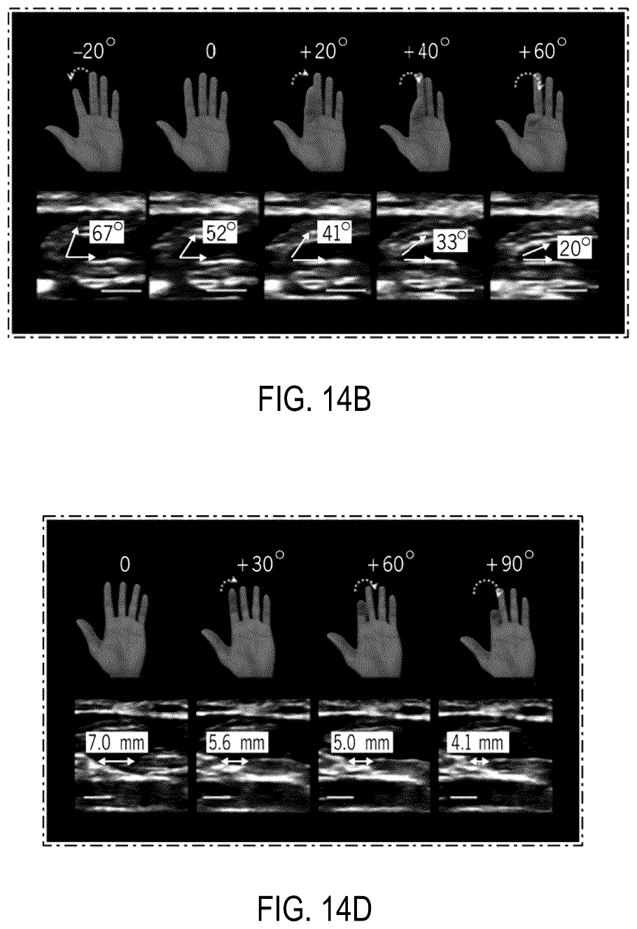

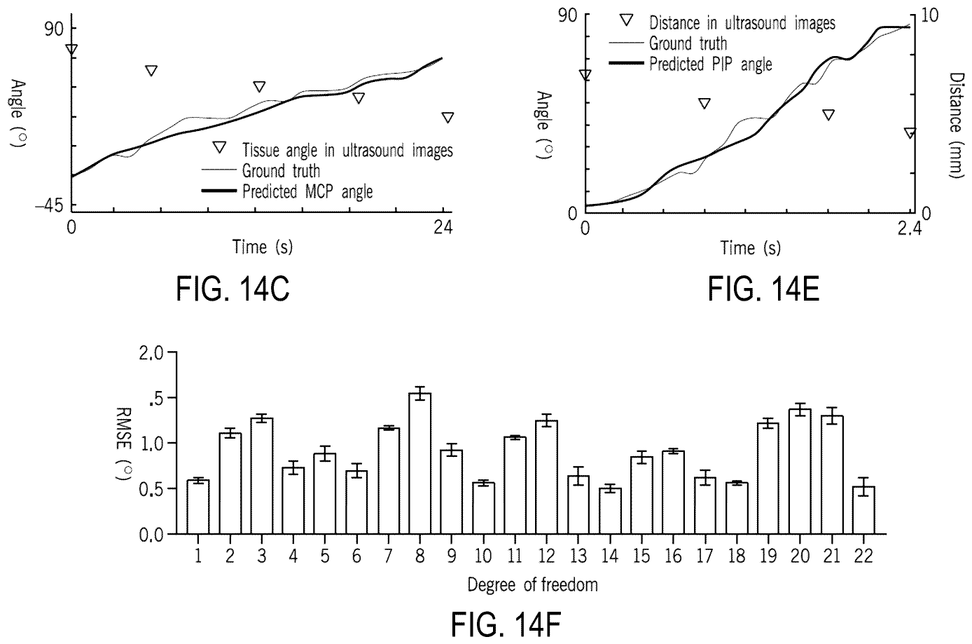

FIG. 14 illustrates the principle and performance of an example of an ultra-dexterous virtual hand. FIG. 14A shows how the 22 DOFs of the hand are correlated to the features of the corresponding sub-regions in the high-resolution ultrasound image. Note that the number of the 22 DOFs is shown in FIG. 10D. FIG. 14B shows a sequence of ultrasound images of the sub-region corresponding to the metacarpophalangeal (MCP) joint angle of the index finger in the flexion and extension motion. The index MCP joint angle increases monotonically with the decrease of a landmark angle in the sub-region. FIG. 14C shows the index MCP joint angles predicted by the wristband compared with the ground truth. FIG. 14D shows a sequence of ultrasound images of the sub-region corresponding to the proximal interphalangeal (PIP) joint angle of the index finger in the flexion motion. The index PIP joint angle increases monotonically with the decrease of the distance between two landmarks in the sub-region. FIG. 14E shows the index PIP joint angles predicted by the wristband compared with the ground truth. FIG. 14F shows the root mean square errors (RMSE) of the 22 DOFs of the hand predicted by the wristband compared with the ground truth. All scale bars indicate 5 mm. Error bars indicate the standard deviation of RMSE (n=5 independent measurements). Sub-regions in the six regions shown in FIG. 13 further characterize the DOFs of individual joints in the corresponding fingers or palm, as shown in FIG. 14A.

Because a joint's DOF is characterized by a unique feature in the corresponding sub-region (e.g., the shapes of the muscles and tendons), the interference among different joints' DOFs is minimal in the high-resolution ultrasound image. The present disclosure implements a compact and efficient AI algorithm for a 22-dimensional output regression model based on the convolutional neural network (CNN), as illustrated in FIG. 15. In FIG. 15, ReLU refers to Rectified Linear Units. To train the CNN, a marker tracking system with multi-view cameras were used to measure the DOFs of the hand as the ground truth values. The trained AI algorithm can quantify all 22 DOFs of the hand's five fingers and palm by analyzing the high-resolution ultrasound images, which have not been used for training. In some examples, the algorithm may be trained on a user-by-user basis. However, in other examples the algorithm may be trained using a larger data set so as to have the ability to predict hand configurations based on imaging data for a wide range of users. In still other examples, the algorithm may be coarsely trained for all users, and subsequently fine-tuned for an individual user.

To validate that the wristband according to the present disclosure can accurately and continuously track the 22 DOFs of the hand, a validation test was performed. In the test, a human subject varied only one DOF of the joints each time while using the wristband to track the 22 DOFs. The change of each DOF is correlated to a distinct shape change in the corresponding sub-region in the ultrasound image, as shown in FIGS. 14B-14E. To further quantify and visualize the relationship between characterizing regions in ultrasound images and degrees of freedom (DOFs), displacement analysis was performed on 22 pairs of ultrasound images. Each pair of images consisted of the start and end frame of one DOF action. Displacement maps (see FIG. 15) visualized changes in ultrasound images during hand motions and can highlight minor displacements that are imperceptible to humans yet noticeable to the computer. While displacement maps were not used as inputs to machine learning models, they can help to see ultrasound images from a view that is closer to computers and enhance the interpretability of the machine learning model.

To further validate that the 22 DOFs tracked by the wristband are quantitatively accurate, the root mean square error between the tracked DOF and the corresponding ground truth measured by the multi-view camera system was calculated. The root mean square errors of the 22 DOFs tracked by the wristband range from 0.53° to 1.37°, indicating a high tracking accuracy (see FIG. 14F). In comparison, the root mean square errors of the DOFs tracked by comparative examples of camera-free hand-free armbands range from 7.35° to 22.58°, and these armbands can only track a limited number (≤5) of DOFs. Furthermore, the processing time of each ultrasound image by the AI algorithm ranges from 0.67 ms to 0.93 ms. This low processing latency enables real-time tracking of the 22 DOFs of the hand motion using the wristband. To validate the general applicability of the wristband, another human subject repeated the test to track the 22 DOFs of the hand.

In addition to the real-time, high-accuracy, and low-latency tracking of the hand's 22 DOFs, the high-resolution ultrasound wristband of the present disclosure also demonstrates exceptional robustness against noise, hysteresis, and drifting effects during hand tracking. These effects constitute limitations for the robustness and performance of comparative examples of camera-free hand-tracking techniques, including strain sensors and EMG sensors.

To evaluate the sensitivity of the wristband to noises, ultrasound image datasets were generated with varying signal-to-noise ratios (i.e., 14 dB, 8 dB, 2 dB, −6 dB) by introducing random Gaussian noises with different standard deviations. The model was trained only on the data without add-on noise. While the AI algorithm is only trained with a noiseless image dataset, the algorithm may be used to quantify the 22 DOFs by analyzing ultrasound image datasets with varying SNRs. The RMSE of the DOF is used to evaluate the sensitivity to noise. The RMSE is smaller than 3.3° when the SNR is higher than 8 dB, and smaller than 11° when the SNR is as low as −6 dB. This low noise sensitivity is because the wristband relies on images and patterns to quantify the DOFs. In contrast, strain sensors and EMG sensors according to comparative examples are known to be sensitive to noise because they rely on linear data curves for hand tracking, which are sensitive to noise.

The drifting effect is often due to the fatigue of hand-related neurons and muscles after long-term usage. To evaluate the drifting effect of the wristband, a human subject exercised the hand for one hour using a hand gripper and a wrist curl. Following the exercise, the ultrasonic wristband was employed to capture the wrist images. No discernible difference can be observed between ultrasound images for the same hand configuration before and after the exercise. Furthermore, the AI algorithm was used to quantify the DOFs of the hand before and after the exercise. The RMSEs of the DOFs are consistently lower than 1.5°, affirming the low drifting effect of the wristband.

Systems, methods, and apparatuses according to the present disclosure allow previously inaccessible applications of the ultra-dexterous virtual hands enabled by the high-resolution ultrasound wristband in virtual reality and robotics. In virtual reality, the wristband can provide a hand-free camera-free human-computer interface that manipulates objects using an ultra-dexterous virtual hand with the 22 DOFs. The ultrasound wristband provides a hand-free camera-free human-computer interface that manipulates objects using an ultra-dexterous virtual hand with the 22 DOFs in virtual reality. For example, the pinch distance can be calculated in real-time to control the size of the photo in virtual reality. In an example implementation, both ‘agile move’ and ‘hold’ of the pinch action are accurately tracked. The ultra-dexterous virtual hand can control the photo with other actions, including rotating the photo around two axes by rotating the palm in two ways and moving the photo forward and backward by bending the four fingers. For example, a human subject can pinch the thumb and index finger to various degrees to accurately manipulate the size of a photo in virtual reality. The wristband can continuously quantify the distance between the two fingertips by tracking the corresponding DOFs, and adjust the photo size accordingly. In contrast, comparative example hand-free camera-free human-computer interfaces such as EMG sensors can only qualitatively detect the pinch gesture and use it as a click command. In addition, the ultra-dexterous virtual hand can stably hold the photo at any specific size, which is also unachievable with comparative example hand-free camera-free human-computer interfaces. The human subject can further rotate the photo around two axes and perform translational movements of the photo by naturally rotating the palm and bending the four fingers. The root mean square errors of the DOFs are consistently lower than 0.95 cm, 0.60°, 1.12°, and 0.85° throughout the four actions in an example experiment. Furthermore, the wristband can also track the hand when performing these actions simultaneously, further demonstrating the intuitive and versatile controls in virtual reality.

In robotics, the wristband can provide a hand-free camera-free human-computer interface that can control the 22 DOFs of a robotic hand. In another example implementation, a human subject controlled a robotic hand to play a desktop basketball game while wearing the wristband. The wristband provided intuitive and versatile controls of a robotic hand with the ultra-dexterous virtual hand enabled by the high-resolution ultrasound wristband. A robotic hand was controlled by the virtual hand in real-time to play a desktop basketball game.

In the demonstration, the human subject bent the MCP joint of the index finger to various angles to quantitatively control the robotic hand. The index finger of the robotic hand bent to the corresponding degrees to press down the ball-shooting pad in real-time. After a few trials of the robotic hand controlled by the human subject, an optimal bending angle of the index finger has been determined to shoot the ball into the hoop.

It was further demonstrated that the human subject can control multiple fingers of the robotic hand to perform more complicated tasks such as playing a plano. To demonstrate the general applicability of the ultra-dexterous virtual hand in robotics, another human subject repeated the control of the robotic hand to play the plano.

### Example Ultrasound System

One example ultrasound array encompassed 256 channels with a central frequency of 18 MHz and a pitch size of 0.2 mm. Design and material selection were optimized using the Krimholtz-Leedom-Matthaei (KLM) model simulation tool (Biosono Inc., Fremont, CA) and summarized in Table 2 above.

The ultrasound array was constructed using high-performance 1-3 composite piezoelectric material. Soft lead zirconate titanate (PZT-5, Del-Piezo Specialties, West Palm Beach, FL) was chosen as the piezoelectric layer, due to its high electromechanical coupling factor and high dielectric constant, which increased the power efficiency of the designed array. The kerf between elements was filled with EPO-TEK 301 (Epoxy Technology, Billerica, MA).

The array's backend was connected to a thin flexible printed circuit board (F-PCB) and a high-attenuation acoustic backing layer, mitigating the ringdown noise in ultrasound signals. The backing layer for the ultrasound probe provided mechanical support to the elements inside the probe that will generate high-frequency vibrations. The backing layer also had strong attenuation of the ultrasound wave to effectively shorten the pulse duration and thus increase the imaging resolution. When the acoustic impedance match between the backing and the piezoelectric material becomes better, the generated pulse will be shorter while the amplitude of the pulse will be lower. Thus, the backing material was carefully in view of this trade-off. In the example design, the KLM model was used to select the design of the backing layer. A 3-mm-thick backing layer made of 3022 E-Solder conductive adhesive (Von Roll, Breitenbach, Switzerland), approximately 24 times the wavelength, was added to the back of the piezoelectric layer. This layer provided mechanical adhesion (adhesive strength: 2030 psi) and electrical connection to the printed circuit board. Additionally, due to the relatively low longitudinal sound velocity (1920 m/s) and high acoustic attenuation coefficient (˜3.67 dB/mm/MHz), this backing layer improved the axial resolution without significantly increasing the size of the array.

To further increase the pressure amplitude and axial resolution of the array, a dual-layer acoustic impedance matching was implemented. The matching layers smoothed the acoustic impedance mismatch between the piezoelectric material and the skin, allowing the acoustic wave from the transducer to smoothly penetrate the skin and the reflected acoustic waves (the returning echo) to smoothly return to the transducer for imaging. The theoretical acoustic impedances of the dual-layer matching are given by the following:

$${Z}_{m 1} = {( {Z}_{p}^{4} {Z}_{w}^{3} )}^{\frac{1}{7}} \qquad ( 1 )$$

$${Z}_{m 2} = {( {Z}_{p}^{1} {Z}_{w}^{6} )}^{\frac{1}{7}} \qquad ( 2 )$$

where Zₘᵢ is the acoustic impedance of matching layer i (i=1 or 2), and Zₚ and Z_{w} are the acoustic impedance of the piezoelectric material and the water, respectively. The 1-3 composite piezoelectric material adopted in this example had an acoustic impedance of 17.1 MRayl and the water had an acoustic impedance of 1.54 MRayl. Based on Equations (1) and (2), Zₘ₁=6.09 MRayl and Zₘ₂=2.17 MRayl. In the example, a quarter-wavelength 2-3-μm silver epoxy composite with a thickness of quarter wavelength was attached to the surface of the piezoelectric layer as the first layer of the matching. The silver epoxy included 2-3-μm silver powders (Fisher Scientific, Hampton, NH) and epoxy mixture included Insulcast 501 and Insulcure 9 (American Safety Technologies, Roseland, NJ). The acoustic impedance of the 2-3-μm silver epoxy is adjustable by changing the ratio of the silver powder. It was adjusted to 6.1 MRayl in this example. Afterwards, a quarter-wavelength parylene C was coated on the array surface as the second layer of the matching. It also provided insulation and protection.

The PZT composite with electrodes in both sides was coated with the 2-3-μm silver epoxy on the top side as the first matching layer. After curing at room temperature for 24 hours, the whole stack was lapped to the designed thickness. To prepare the PZT element of each channel, the composite piezoelectric material was further cut by a sub-scratch-dicing process (Tcar 864-1, Thermocarbon, Casselberry, FL). Subsequently, the prepared PZT composite with 256 separated channels was bonded and glued to the designed F-PCB. The top side of the material and the ground pads of the F-PCB were then sputtered with a Cr/Au (50/100 nm) electrode by a sputtering system (NSC-3000 Sputter Coater, Nano-Master, Inc., Austin, TX) for ground connection. The E-solder 3022 backing was glued to the other side of the F-PCB as the backing layer. Finally, the amount of Parylene C film was coated on the surface of the materials as the second matching layer.

To characterize the performance of the fabricated ultrasound array, KLM modeling, impedance spectrum measurements, and pulse-echo tests were conducted. The electric impedance of the piezoelectric element was 211Ω at 17.7 MHz with a proportional −6-dB bandwidth of 61.6%. Modeling results and experiment results matched well. The measured center frequency of all elements was 16.9 MHz±1.1 MHz, bandwidth was 63.4%±3.2%, and a signal amplitude difference was less than −6 dB.

The performance of each element in the fabricated array (center frequency, bandwidth, signal amplitude, and level of crosstalk) was characterized by connecting the array to the 256-channel Vantage system (Verasonics, Inc., Kirkland, WA). The pulse-echo signal (1-cycle pulse, 20 Vpp) of each element was acquired and analyzed to determine the center frequency, bandwidth, and signal amplitude. The electrical impedance of the fabricated array was assessed in air using an impedance analyzer (E4990A, Keysight, Santa Rosa, CA).

The 256-channel Vantage system (Verasonics, Inc, Kirkland, WA) was used for imaging. Radio-frequency signals received by the ultrasound probe were acquired, digitized, and post-processed in real-time by the Vantage system. Ultrasound imaging was performed using a line-focused beamforming mode with an electrical focus at 1 cm depth. The imaging speed was 90 frames per second. All imaging algorithms and image post-processing were adapted from the algorithm packages of the Verasonics Vantage system. The ultrasound imaging speed can be improved to over 500 frame rates by using the plane wave beamforming mode. Thus, imaging speed does not limit the tracking rate of the ultrasonic wristband.

A multi-purpose multi-tissue phantom (MODEL 040GSE, CIRS Inc., Norfolk, VA, USA) was used in the phantom imaging test. The resolution section of the phantom was made of 80 μm diameter nylon monofilament wires. Their axial separations were 4, 3, 2, 1, 0.5 and 0.25 mm, and lateral separations were 4, 3, 2, 1, 0.5 and 0.25 mm. The resolution of the ultrasound imaging probe was determined by measuring the full beam width at half maximum of single wire. The measured resolutions were also confirmed by the minimum distance between distinguishable phantom wires. The contrasts of hyperechoic reflectors were 3, 6, and 15 dB, from left to right. The contrasts of anechoic reflectors were −9, −6, and −3 dB, from left to right. To quantify the image contrast, signals from the reflector-free regions at the same depth (4 cm) were taken as the background signals (Sb). Signals from each reflector were taken as reflector signals (Sr). The imaging contrasts were calculated according to the following:

$$Imaging contrast = 20 \times {log}_{10} ( \frac{\int {S}_{r}}{\int {S}_{b}} ) \qquad ( 3 )$$

An 8-camera hand motion tracking system (Motion Analysis, Rohnert Park, CA) with 25 markers was used to capture hand motions in real time at a frame rate of 100 Hz (up to 810 Hz). 3D positions of each marker were acquired and used to calculate the bending angles of 22 DOFs. To synchronize the hand tracking data and ultrasound images acquired from the Verasonics Vantage system, a linear interpolation was performed on the hand tracking data to match the time stamp of each ultrasound image. All experiments were approved by the Massachusetts Institute of Technology Committee on the Use of Humans as Experimental Subjects. Two intact human participants (1 male 1 female, aged 28 and 23 years) with no reported neurological disorders were recruited in the experiment.

Before being fed into the machine learning model, all ultrasound images were pre-processed. All images were sampled to have a size of 250 by 150 pixels and normalized to have a value from 0 to 1. FIG. 14B illustrates the detailed architecture of the CNN-based 22-dimensional output regression model according to the present disclosure. Feature maps of each image were first extracted by two layers of convolutional neural network without pre-trained weights followed by a 2-by-2 Max pool layer. Next, two fully connected layers with ReLU as the activation functions were used to achieve 22 outputs. The learning objective was to minimize the mean square error of all 22 outputs. Optimization of the model was performed using an adaptive moment estimation (ADAM) optimizer in a batch size of 64 and 50 epochs. Max pooling is used to reduce the dimensionality of the data by passing only the locally highest activations.

The models in this example were implemented on a Windows 11 computer equipped with an Intel i9-13900k processing unit and NVIDIA RTX 4090 graphics processing unit. The model development platform was Python 3.10 with the PyTorch (version 2.10; pytorch.org) and CUDA (version 12.1) deep-learning framework. For analyzing the effects of hysteresis, the similarities between ultrasound images were computed using the structural similarity index function from MATLAB R2022a (Mathworks, Natick, MA).

Virtual reality demonstration was developed using Unity 2021.3.29. The ultrasound images acquired from the Verasonics Vantage system were processed in Python to predict the value of 22 DOFs. The outputs from the Python script were returned to Unity C # to control the object in virtual space in real time. For robotic hand control, a programmable 6-DOF (5 for fingers and 1 for the wrist) robotic hand (uHandPi, Hiwonder, Shenzhen) was used. The communication between the robotic hand and the computer was via the STM32 controller and the applicable programing interface provided by the manufacturer. The controlling scripts were developed based on the script packages from the manufacturer and with the help from its customer service.

To better analyze the anatomical meaning of each ultrasound region, magnetic resonance imaging (MRI) was performed to capture cross-sectional images at the same wrist position and aligned MRI images with ultrasound images. Results showed consistency between characterizing regions of 22 DOFs in the ultrasound image and the anatomy locations in the magnetic resonance image (MRI).

MRI of the wrist were obtained on a Three Tesla, Siemens Magnetom, Prisma MRI scanner (Siemens Healthcare GmbH). A home built, 100 mm diameter, receive-only, circular surface coil was used for improving the signal noise ratio over the available RF coils and to eliminate signal from the contra-lateral wrist and other anatomy. Seventeen, 3 mm slices were acquired with a fast spin echo (FSE) pulse sequence with TR=951 ms, TE=15 ms, a turbo factor of 6, and a refocusing angle of 150 degrees. In-plane pixel size was 0.22 mm by 0.22 mm with a slice thickness of 3 mm. Total scan time was just over 6 minutes. Slices were taken orthogonally to the axis of the ulna and radius of a volunteer. The distribution of characterizing regions in the ultrasound images was consistent with the anatomical distribution of tendons and muscles responsible for controlling each DOF. For example, the FDS tendon of the middle finger, which is used in flexing both middle and proximal phalanges, is slightly above the FDS tendon of the index finger. FDP tendons of the index to the little fingers are deeper, distributed from left to right.

EMG signals were generated from using an open-access online dataset and were plotted using MATLAB. No additional signal processing was performed on the data.

To compute the displacement maps between two ultrasound images, a customized script based on 2D cross-correlation was developed in MATLAB. Ultrasound images were first resized to be 200 by 100 pixels and normalized to 0 to 1. To compute the displacement, the cross-correlation was performed using a 15-by-15-pixel window shifting with a 1-pixel step. The distance between the maximum value in correlation results and the center is the displacement. Only the displacement with a correlation value larger than 0.5 is plotted. While both lateral and axial displacements are available, only axial displacement maps were selected as representative results.

The hand is the most dexterous and versatile manipulative organ in the human body. As shown in the present disclosure, high-resolution ultrasound imaging of tendons and muscles in the wrist can continuously and accurately track all 22 DOFs of the hand's five fingers and palm in real-time. Similarly, wearable ultrasound patches may image tendons, muscles, and ligaments in other parts of the body to track other DOFs of the full human body. A set of such ultrasound bands and patches could provide a camera-free and wearable strategy for continuously and accurately tracking the configurations and motions of the full human body in real-time during daily activities.

Other examples and uses of the disclosed technology will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

1. A body motion tracking system, the system comprising:
one or more ultrasound systems configured to be worn by a subject and to acquire ultrasound data during motion of a first location on the subject;
one or more inertia measurement unit (IMU) systems configured to be worn on a second location on the subject and to measure inertia data associated with motion of the second location; and
a control system configured to integrate the ultrasound data and the inertia data to generate position data, the position data characterizing a motion of one of the first or second location on the subject.

2. The system of claim 1, further comprising a plurality of IMU systems configured to be placed on at least one limb of the subject or an IMU system configured to be placed on a trunk of the subject, and wherein the position data characterizes a motion of at least one of the at least one limb or the whole body of the subject.

3. The system of claim 1, further comprising one or more cameras configured to calibrate the inertia data with the position data.

4. The system of claim 1, wherein the ultrasound system comprises an acoustic couplant.

5. The system of claim 4, wherein the acoustic couplant comprises at least one of an adhesive, a hydrogel, a polymer, an elastomer, or a grease.

6. A human motion tracking system, comprising:
a wearable ultrasound system configured to acquire ultrasound data from a first structure of a body of a wearer;
a wearable inertial measurement unit (IMU) system configured to measure inertia data associated with a second structure of the body of the wearer; and
one or more control systems storing a first algorithm and a second algorithm;
wherein the first algorithm is configured to receive the ultrasound data from the wearable ultrasound system and determine a pose of the wearer's first structure based on the ultrasound data;
wherein the second algorithm is configured to receive the inertia data from the IMU system and determine a position of the wearer's second structure based on the inertia data; and
wherein the one or more control systems are further configured to integrate the pose of the wearer's first structure with the position of the wearer's second structure to generate position data of an extended region of the body of the wearer comprising the first structure and the second structure.

7. The system of claim 6, wherein the first structure of the wearer is a wrist of the wearer.

8. The system of claim 7, wherein the second structure of the wearer is a forearm of the wearer.

9. The system of claim 6, wherein the pose of the wearer is a hand configuration of the wearer.

10. The system of claim 9, wherein the hand configuration of the wearer includes information regarding more than five degrees of freedom of the hand.

11. The system of claim 9, wherein the hand configuration of the wearer includes information regarding twenty-two degrees of freedom of the hand.

12. The system of claim 6, wherein the one or more control systems comprise a local control system and a central control system, the local control system being coupled to the ultrasound system and in wireless communication with the central control system, and the central control system being in wireless communication with the ultrasound system and wireless communication with the IMU system; and
wherein the local control system stores the first algorithm, and the central control system stores the second algorithm.

13. The system of claim 6, wherein the wearable ultrasound system further comprises an ultrasound couplant configured to couple the wearable ultrasound system to a surface of the body of the wearer.

14. The system of claim 13, wherein the ultrasound couplant includes a hydrogel or other elastic solid gels.

15. The system of claim 6, wherein the wearable ultrasound system includes:
a backing layer;
a circuit layer;
a piezoelectric layer; or
a matching layer.

16. The system of claim 6, wherein the wearable ultrasound system is at least one of a wristband or a patch.

17. A method of determining a pose of a portion of a user, the method comprising:
imaging an internal structure of a body of the user using a wearable ultrasound system;
inputting ultrasound data from the wearable ultrasound system to a machine learning algorithm;
determining a pose of a first structure of the user by the machine learning algorithm, based on the ultrasound data;
measuring inertia data using a wearable IMU system coupled to the second structure of the user;
inputting the inertia data from the IMU system to an algorithm;
determining a pose of the second structure of the user by the algorithm, based on the inertia data; and
integrating the pose of the first structure of the user and the pose of the second structure of the user to determine the pose of the portion of the user.

18. The method of claim 17, wherein the first structure is a wrist of the use, and wherein the second structure is a forearm of the user.

19. The method of claim 18, wherein the pose of the first structure of the user is a hand configuration of the user.

20. The method of claim 17, wherein the machine learning algorithm was trained on training data comprising sensor data measured from a wearable ultrasound system paired with camera data measured by a camera.
